(19) United States
(12) Patent Application Publication
(10) Pub. No.: US 2023/0024846 A1
(43) Pub. Date: Jan. 26, 2023

(54) CROP YIELD PREDICTION METHOD AND SYSTEM

(71) Applicant: Jilin University, Changchun City (CN)

(72) Inventors: Shengbo CHEN, Changchun City (CN); Bingxue ZHU, Changchun City (CN); Peng LU, Changchun City (CN)

(21) Appl. No.: 17/834,161

(22) Filed: Jun. 7, 2022

(30) Foreign Application Priority Data
Jul. 26, 2021 (CN) 202110845851.6

(51) Int. Cl. G01N 21/31; G01N 33/00

(52) U.S. Cl. CPC G01N 21/31; G01N 33/0098; G06Q 50/02

(57) ABSTRACT

A crop yield prediction method and system. The method includes: obtaining a test normalized difference vegetation index and test meteorological data of a to-be-tested area; and inputting the test normalized difference vegetation index and the test meteorological data into a hierarchical linear regression model, to obtain a predicted yield of the to-be-tested area; where a method for determining the hierarchical linear regression model is: obtaining a training normalized difference vegetation index of a crop planting area; obtaining training meteorological data and measured yield data of the crop planting area; constructing a first regression equation and a second regression equation, where dependent variables of the second regression equation are a slope and an intercept of the first regression equation; and inputting the training normalized difference vegetation index and the measured yield data into the first regression equation, and inputting the training meteorological data into the second regression equation.

### CROSS-REFERENCE TO RELATED APPLICATIONS

This application claims priority to Chinese Patent Application No. 202110845851.6, filed Jul. 26, 2021. The entire disclosure of the above application is incorporated herein by reference.

### TECHNICAL FIELD

The present disclosure relates to the field of crop yield prediction, and in particular, to a crop yield prediction method and system.

### BACKGROUND

Predicting large-scale crop yields is vital to ensure national food security, economics, and politics. However, the traditional field manual measurement method cannot accurately predict the yields of crops with a large planting area. Predicting large-area crop yields by remote sensing is a non-contact, economical, and efficient yield measurement method. Real-time reflectance data of large-scale field crops is obtained using aerospace sensors, a model of yield ground truths and spectral information is established, and then field crop yield information is obtained using the model. This can greatly reduce the workload of field manual measurement, shorten data collection time, and improve economic efficiency.

In terms of model types, regression models can be divided into linear and nonlinear models. A linear model is usually easy to build, but is less variable and less accurate than a nonlinear model. The nonlinear model is more complex and adaptable, but it usually needs to be trained with a large amount of data. As such, when the nonlinear model is used for crop yield prediction, a large amount of information needs to be collected, which makes crop yield prediction inconvenient.

### SUMMARY

In order to overcome the deficiencies of the prior art, the present disclosure is intended to provide a crop yield prediction method and system, to make crop yield prediction much easier.

In order to achieve the above objective, the present disclosure provides the following technical solutions:

A crop yield prediction method includes:

obtaining a test normalized difference vegetation index and test meteorological data of a to-be-tested area; and

inputting the test normalized difference vegetation index and the test meteorological data into a hierarchical linear regression model, to obtain a predicted yield of the to-be-tested area;

where

a method for determining the hierarchical linear regression model is:

obtaining a training normalized difference vegetation index of a crop planting area;

obtaining training meteorological data and measured yield data of the crop planting area;

constructing a first regression equation and a second regression equation, where dependent variables of the second regression equation are a slope and an intercept of the first regression equation; and

inputting the training normalized difference vegetation index and the measured yield data into the first regression equation, and the training meteorological data into the second regression equation to train the first regression equation and the second regression equation, and determining the trained first regression equation as the hierarchical linear regression model.

Preferably, the obtaining a training normalized difference vegetation index of a crop planting area includes:

obtaining remote sensing image data of the crop planting area;

calculating a spectral reflectance based on the remote sensing image data; and

performing band calculation on the spectral reflectance to obtain the training normalized difference vegetation index.

Preferably, the remote sensing image data is Landsat image data; and bands of the Landsat image data include blue band, green band, red band, and near-infrared band.

Preferably, a formula for performing band calculation on the spectral reflectance is:

NDVI=(ρ_{NIR}−ρ_{R})/(ρ_{NIR}+ρ_{R}), where

ρ_{NIR} is a spectral reflectance of near-infrared band; PR is a spectral reflectance of red band, and NDVI is the training normalized difference vegetation index.

Preferably, a formula of the first regression equation is:

Yᵢⱼ=β₀ⱼ+β₁ⱼ×NVDIᵢ+eᵢⱼ, where

β₀ⱼ is the intercept of the first regression equation, β₁ⱼ is the slope of the first regression equation, eᵢⱼ is a random error of the first regression equation, Yᵢⱼ is the i-th predicted yield, NDVIᵢ is the i-th normalized difference vegetation index in the training normalized difference vegetation indices, and j is a numerical subscript.

Preferably, a formula of the second regression equation is:

β₀ⱼ=γ₀₀+γ₀₁×RAD+γ₀₂×Tₘₐₓ+γ₀₃×Tₘᵢₙ+γ₀₄×PRE+μ₀ⱼ;

β₁ⱼ=γ₁₀+γ₁₁×RAD+γ₁₂×Tₘₐₓ+γ₁₃×Tₘᵢₙ+γ₁₄×PRE+μ₁ⱼ, where

γ₀₀ is a first intercept of the second regression equation, γ₁₀ is a second intercept of the second regression equation, RAD is average sunshine duration in the training meteorological data, γ₀₁ is a first slope of the average sunshine duration, γ₁₁ is a second slope of the average sunshine duration, Tₘₐₓ is average daily maximum temperature in the training meteorological data, you is a first slope of the average daily maximum temperature, γ₁₂ is a second slope of the average daily maximum temperature, Tₘᵢₙ is average daily minimum temperature in the training meteorological data, γ₀₃ is a first slope of the average daily minimum temperature, γ₁₃ is a second slope of the average daily minimum temperature, PRE is average daily precipitation in the training meteorological data, γ₀₄ is a first slope of the average daily precipitation, γ₁₄ is a second slope of the average daily precipitation, μ₀ⱼ is a first random error of the second regression equation, and is a second random error of the second regression equation.

Preferably, the crop in the to-be-tested area is corn.

Preferably, the corn is in the grain filling stage.

Preferably, the obtaining training meteorological data of the crop planting area includes:

obtaining a daily value data set of surface climate data, where the daily value data set of surface climate data includes daily maximum temperature, daily minimum temperature, daily precipitation, and sunshine duration of the to-be-tested area; and

calculating the training meteorological data based on the daily value data set of surface climate data, where the training meteorological data includes average daily maximum temperature, average daily minimum temperature, average daily precipitation, and average sunshine duration.

A crop yield prediction system includes:

a test data obtaining module, configured to obtain a test normalized difference vegetation index and test meteorological data of a to-be-tested area; and

a prediction module, configured to input the test normalized difference vegetation index and the test meteorological data into a hierarchical linear regression model, to obtain a predicted yield of the to-be-tested area; where

the prediction module includes:

a first obtaining module, configured to obtain a training normalized difference vegetation index of a crop planting area;

a second obtaining module, configured to obtain training meteorological data and measured yield data of the crop planting area;

a construction module, configured to construct a first regression equation and a second regression equation, where dependent variables of the second regression equation are a slope and an intercept of the first regression equation; and

a training module, configured to input the training normalized difference vegetation index and the measured yield data into the first regression equation, and the training meteorological data into the second regression equation to train the first regression equation and the second regression equation, and determine the trained first regression equation as the hierarchical linear regression model.

According to the specific embodiments provided by the present disclosure, the present disclosure discloses the following technical effects:

The present disclosure provides a crop yield prediction method and system. The method includes: obtaining a test normalized difference vegetation index and test meteorological data of a to-be-tested area; and inputting the test normalized difference vegetation index and the test meteorological data into a hierarchical linear regression model, to obtain a predicted yield of the to-be-tested area; where a method for determining the hierarchical linear regression model is: obtaining a training normalized difference vegetation index of a crop planting area; obtaining training meteorological data and measured yield data of the crop planting area; constructing a first regression equation and a second regression equation, where dependent variables of the second regression equation are a slope and an intercept of the first regression equation; and inputting the training normalized difference vegetation index and the measured yield data into the first regression equation, and the training meteorological data into the second regression equation to train the first regression equation and the second regression equation, and determining the trained first regression equation as the hierarchical linear regression model. In the present disclosure, a model relationship between crop yields and spectral and meteorological data is constructed through hierarchical linear regression equations, to predict crop yields in unknown areas based on spectral information and meteorological data. The present disclosure combines two types of linear regression models, can enhance model adaptability with relatively less data, and does not require massive information collection in the early stage, making crop yield prediction much easier.

### BRIEF DESCRIPTION OF THE DRAWINGS

To describe the embodiments of the present disclosure or the technical solutions in the related art more clearly, the accompanying drawings required in the embodiments are briefly introduced below. Obviously, the accompanying drawings described below are only some embodiments of the present disclosure. A person of ordinary skill in the art may further obtain other accompanying drawings based on these accompanying drawings without creative efforts.

FIG. 1 is a flowchart of a crop yield prediction method according to an embodiment of the present disclosure.

FIG. 2 is a module connection diagram of a crop yield prediction system according to an embodiment of the present disclosure.

### DETAILED DESCRIPTION OF THE EMBODIMENTS

The technical solutions in the embodiments of the present disclosure will be described below clearly and completely with reference to the accompanying drawings in the embodiments of the present disclosure. Apparently, the described embodiments are merely some rather than all of the embodiments of the present disclosure. All other embodiments obtained by a person of ordinary skill in the art based on the embodiments of the present disclosure without creative efforts shall fall within the protection scope of the present disclosure.

To make the above-mentioned objective, features, and advantages of the present disclosure clearer and more comprehensible, the present disclosure will be further described in detail below in conjunction with the accompanying drawings and specific embodiments.

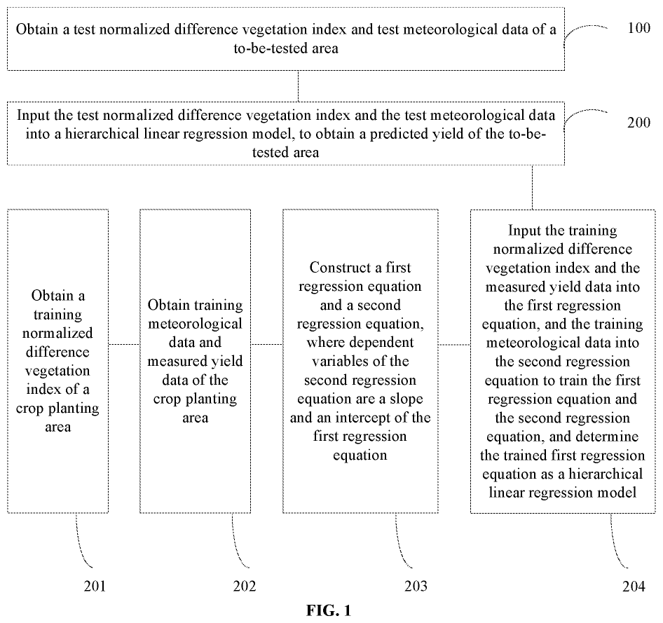

FIG. 1 is a flowchart of a crop yield prediction method according to an embodiment of the present disclosure. As shown in FIG. 1, the crop yield prediction method includes:

Step 100: obtain a test normalized difference vegetation index and test meteorological data of a to-be-tested area; and

Step 200: input the test normalized difference vegetation index and the test meteorological data into a hierarchical linear regression model, to obtain a predicted yield of the to-be-tested area.

A method for determining the hierarchical linear regression model is:

Step 201: obtain a training normalized difference vegetation index of a crop planting area;

Step 202: obtain training meteorological data and measured yield data of the crop planting area;

Step 203: construct a first regression equation and a second regression equation, where dependent variables of the second regression equation are a slope and an intercept of the first regression equation; and

Step 204: input the training normalized difference vegetation index and the measured yield data into the first regression equation, and the training meteorological data into the second regression equation to train the first regression equation and the second regression equation, and determine the trained first regression equation as the hierarchical linear regression model.

Preferably, the crop in the to-be-tested area is corn. The corn is in the grain filling stage.

Optionally, this embodiment aims to provide a method for predicting corn yields in a large area under multiple weather conditions, to accurately predict corn yields at unknown areas.

Preferably, the obtaining a training normalized difference vegetation index of a crop planting area includes:

obtaining remote sensing image data of the crop planting area;

calculating a spectral reflectance based on the remote sensing image data; and

performing band calculation on the spectral reflectance to obtain the training normalized difference vegetation index.

Preferably, the remote sensing image data is Landsat image data; and bands of the Landsat image data include blue band, green band, red band, and near-infrared band.

Specifically, the remote sensing data is Landsat image data, which covers nine bands, including blue, green, red, and near-infrared bands, a wavelength ranges from 0.43 to 1.38, and multispectral band spatial resolution is 30 meters.

Preferably, the obtaining training meteorological data of the crop planting area includes:

obtaining a daily value data set of surface climate data, where the daily value data set of surface climate data includes daily maximum temperature, daily minimum temperature, daily precipitation, and sunshine duration of the to-be-tested area; and

calculating the training meteorological data based on the daily value data set of surface climate data, where the training meteorological data includes average daily maximum temperature, average daily minimum temperature, average daily precipitation, and average sunshine duration.

Optionally, the meteorological data comes from site meteorological information published at the China Meteorological Data website, and the data set is the daily value data set of China surface climate data (V3.0), including daily maximum temperature, daily minimum temperature, daily precipitation, and sunshine duration.

In an optional embodiment, the measured yield data comes from field yield data.

Preferably, a formula for performing band calculation on the spectral reflectance is: NDVI=(ρ_{NIR}−ρ_{R})/(ρ_{NIR}+ρ_{R}), where

ρ_{NIR} is a spectral reflectance of near-infrared band; ρ_{R} is a spectral reflectance of red band, and NDVI is the training normalized difference vegetation index.

When a light source illuminates the surface of an object, the object selectively reflects electromagnetic waves of different wavelengths. The spectral reflectance is a ratio of the luminous flux reflected by the object at a band to the luminous flux received by the object, and is an essential property of the object surface. The spectral reflectance is the representation of color by the object itself, which not only records the color information of the object, but also represents the surface material of the object.

In this embodiment, the first regression equation and the second regression equation are combined to form a hierarchical linear regression model. When data is present in different levels, variables at the first layer are used to construct a regression equation, and then an intercept and a slope in the equation are used as dependent variables, and variables at the second layer are used as independent variables, to construct two new equations. In this way, influence of variables at different layers on dependent variables can be explored. Because the intercept and slope in the first-layer regression equation are used as random variables in the second-layer regression equation, this is also called “regression of regression”.

Preferably, a formula of the first regression equation is: Yᵢⱼ=β₀ⱼ+β₁ⱼ×NVDIᵢ+eᵢⱼ, where

β₀ⱼ is the intercept of the first regression equation, β₁ⱼ is the slope of the first regression equation, eᵢⱼ is a random error of the first regression equation, Yᵢⱼ is the i-th predicted yield, NDVIᵢ is the i-th training normalized difference vegetation index, and j is a numerical subscript.

Specifically, the first regression equation constitutes the first layer of the hierarchical linear regression model, and the first layer is similar to an ordinary least squares (OLS) regression model, and includes an independent variable remote sensing parameter (NDVI) and a dependent variable Y (yield).

Preferably, a formula of the second regression equation is as follows:

β₀ⱼ=γ₀₀+γ₀₁×RAD+γ₀₂×Tₘₐₓ+γ₀₃×Tₘᵢₙ+γ₀₄×PRE+μ₀ⱼ;

β₁ⱼ=γ₁₀+γ₁₁×RAD+γ₁₂×Tₘₐₓ+γ₁₃×Tₘᵢₙ+γ₁₄×PRE+μ₁ⱼ, where

γ₀₀ is a first intercept of the second regression equation, γ₁₀ is a second intercept of the second regression equation, RAD is average sunshine duration in the training meteorological data, γ₀₁ is a first slope of the average sunshine duration, γ₁₁ is a second slope of the average sunshine duration, Tₘₐₓ is average daily maximum temperature in the training meteorological data, you is a first slope of the average daily maximum temperature, γ₁₂ is a second slope of the average daily maximum temperature, Tₘᵢₙ is average daily minimum temperature in the training meteorological data, γ₀₃ is a first slope of the average daily minimum temperature, γ₁₃ is a second slope of the average daily minimum temperature, PRE is average daily precipitation in the training meteorological data, γ₀₄ is a first slope of the average daily precipitation, γ₁₄ is a second slope of the average daily precipitation, μ₀ⱼ is a first random error of the second regression equation, and μ₁ⱼ is a second random error of the second regression equation.

Specifically, the second-layer equation is as follows:

βₘⱼ=γₘ₀+γₘ₁×RAD+γₘ₂×Tₘₐₓ+γₘ₃×Tₘᵢₙ+γₘ₄×PRE+μₘⱼ. When m is equal to 1, the dependent variable is the slope in the first-layer model; when m is equal to 0, the dependent variable is equal to the intercept in the first-level model. Independent variables in the second-layer model are meteorological parameters (RAD, PRE, Tₘₐₓ, Tₘᵢₙ). Quantities to be solved are γₘ₁ to γₘ₄.

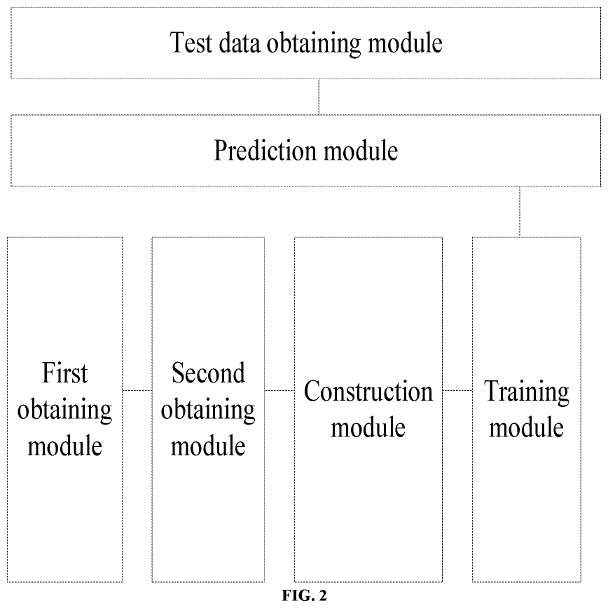

This embodiment also provides a crop yield prediction system. FIG. 2 is a module connection diagram of a crop yield prediction system according to an embodiment of the present disclosure. As shown in FIG. 2, the system includes:

a test data obtaining module, configured to obtain a test normalized difference vegetation index and test meteorological data of a to-be-tested area; and

a prediction module, configured to input the test normalized difference vegetation index and the test meteorological data into a hierarchical linear regression model, to obtain a predicted yield of the to-be-tested area. The hierarchical linear regression model is determined by a regression model construction module.

The regression model construction module includes:

a first obtaining module, configured to obtain a training normalized difference vegetation index of a crop planting area;

a second obtaining module, configured to obtain training meteorological data and measured yield data of the crop planting area;

a construction module, configured to construct a first regression equation and a second regression equation, where dependent variables of the second regression equation are a slope and an intercept of the first regression equation; and

a training module, configured to input the training normalized difference vegetation index and the measured yield data into the first regression equation, and the training meteorological data into the second regression equation to train the first regression equation and the second regression equation, and determine the trained first regression equation as the hierarchical linear regression model.

Specifically, the first obtaining module includes:

a first obtaining unit, configured to obtain remote sensing image data of a corn planting area;

a first calculation unit, configured to calculate a spectral reflectance based on the remote sensing image data; and

a second calculation unit, configured to perform band calculation on the spectral reflectance to obtain the training normalized difference vegetation index.

The present disclosure has the following beneficial effects:

Based on hierarchical linear regression modeling, the present disclosure combines spectral information of the corn grain filling stage obtained by sensors with local meteorological data, and constructs a model relationship between corn yields and spectral and meteorological data through hierarchical linear regression equations, so as to predict corn yields in unknown areas based on the spectral information and meteorological data. The present disclosure can enhance model adaptability with relatively less data, and does not require massive information collection in the early stage, making crop yield prediction much easier. Moreover, compared with a pure linear or nonlinear model, the present disclosure can achieve more accurate prediction results.

Each embodiment of the present specification is described in a progressive manner, each embodiment focuses on the difference from other embodiments, and mutual reference may be made between the same and similar parts. Since the system disclosed in an embodiment corresponds to the method disclosed in another embodiment, the description is relatively simple, and reference can be made to the method description.

Specific examples are used herein to explain the principles and embodiments of the present disclosure. The foregoing description of the embodiments is merely intended to help understand the method of the present disclosure and its core ideas; besides, various modifications may be made by a person of ordinary skill in the art to specific embodiments and the scope of application in accordance with the ideas of the present disclosure. In conclusion, the content of the present specification shall not be construed as limitations to the present disclosure.

What is claimed is:

1. A crop yield prediction method, comprising:
obtaining a test normalized difference vegetation index and test meteorological data of a to-be-tested area; and
inputting the test normalized difference vegetation index and the test meteorological data into a hierarchical linear regression model, to obtain a predicted yield of the to-be-tested area; wherein
a method for determining the hierarchical linear regression model is:
obtaining a training normalized difference vegetation index of a crop planting area;
obtaining training meteorological data and measured yield data of the crop planting area;
constructing a first regression equation and a second regression equation, wherein dependent variables of the second regression equation are a slope and an intercept of the first regression equation; and
inputting the training normalized difference vegetation index and the measured yield data into the first regression equation, and the training meteorological data into the second regression
equation to train the first regression equation and the second regression equation, and determining the trained first regression equation as the hierarchical linear regression model.

2. The crop yield prediction method according to claim 1, wherein the obtaining a training normalized difference vegetation index of a crop planting area comprises:
obtaining remote sensing image data of the crop planting area;
calculating a spectral reflectance based on the remote sensing image data; and
performing band calculation on the spectral reflectance to obtain the training normalized difference vegetation index.

3. The crop yield prediction method according to claim 2, wherein the remote sensing image data is Landsat image data; and bands of the Landsat image data comprises blue band, green band, red band, and near-infrared band.

4. The crop yield prediction method according to claim 2, wherein a formula for performing band calculation on the spectral reflectance is:
NDVI=(ρ_{NIR}−ρ_{R})/(ρ_{NIR}+ρ_{R}), wherein
ρ_{NIR} is a spectral reflectance of near-infrared band; ρ_{R} is a spectral reflectance of red band, and NDVI is the training normalized difference vegetation index.

5. The crop yield prediction method according to claim 1, wherein a formula of the first regression equation is:
Yᵢⱼ=β₀ⱼ+β₁ⱼ×NVDIᵢ+eᵢⱼ, wherein
β₀ⱼ is the intercept of the first regression equation, β₁ⱼ is the slope of the first regression equation, eᵢⱼ is a random error of the first regression equation, Yᵢⱼ is the i-th predicted yield, NDVIᵢ is the i-th normalized difference vegetation index in the training normalized difference vegetation indices, and j is a numerical subscript.

6. The crop yield prediction method according to claim 5, wherein a formula of the second regression equation is:
β₀ⱼ=γ₀₀+γ₀₁×RAD+γ₀₂×Tₘₐₓ+γ₀₃×Tₘᵢₙ+γ₀₄×PRE+μ₀ⱼ;
β₁ⱼ=γ₁₀+γ₁₁×RAD+γ₁₂×Tₘₐₓ+γ₁₃×Tₘᵢₙ+γ₁₄×PRE+μ₁ⱼ, wherein
γ₀₀ is a first intercept of the second regression equation, γ₁₀ is a second intercept of the second regression equation, RAD is average sunshine duration in the training meteorological data, γ₀₁ is a first slope of the average sunshine duration, γ₁₁ is a second slope of the average sunshine duration, Tₘₐₓ is average daily maximum temperature in the training meteorological data, γ₀₂ is a first slope of the average daily maximum temperature, γ₁₂ is a second slope of the average daily maximum temperature, Tₘᵢₙ is average daily minimum temperature in the training meteorological data, γ₀₃ is a first slope of the average daily minimum temperature, γ₁₃ is a second slope of the average daily minimum temperature, PRE is average daily precipitation in the training meteorological data, γ₀₄ is a first slope of the average daily precipitation, γ₁₄ is a second slope of the average daily recipitation, μ₀ⱼ is a first random error of the second regression equation, and μ₁ⱼ is a second random error of the second regression equation.

7. The crop yield prediction method according to claim 1, wherein the crop in the to-be-tested area is corn.

8. The crop yield prediction method according to claim 7, wherein the corn is in the grain filling stage.

9. The crop yield prediction method according to claim 1, wherein the obtaining training meteorological data of a crop planting area comprises:
obtaining a daily value data set of surface climate data, wherein the daily value data set of surface climate data comprises daily maximum temperature, daily minimum temperature, daily precipitation, and sunshine duration of the to-be-tested area; and
calculating the training meteorological data based on the daily value data set of surface climate data, wherein the training meteorological data comprises average daily maximum temperature, average daily minimum temperature, average daily precipitation, and average sunshine duration.

10. A crop yield prediction system, comprising:
a test data obtaining module, configured to obtain a test normalized difference vegetation index and test meteorological data of a to-be-tested area; and
a prediction module, configured to input the test normalized difference vegetation index and the test meteorological data into a hierarchical linear regression model, to obtain a predicted yield of the to-be-tested area; wherein
the prediction module comprises:
a first obtaining module, configured to obtain a training normalized difference vegetation index of a crop planting area;
a second obtaining module, configured to obtain training meteorological data and measured yield data of the crop planting area;
a construction module, configured to construct a first regression equation and a second regression equation; and
a training module, configured to train the first regression equation based on the training normalized difference vegetation index and the measured yield data, and train the second regression equation based on the training meteorological data, so as to obtain the hierarchical linear regression model, wherein dependent variables of the second regression equation are a slope and an intercept of the first regression equation.
